(19) United States
(12) Patent Application Publication
(10) Pub. No.: US 2012/0246863 A1
(43) Pub. Date: Oct. 4, 2012

(54) CONTROL SYSTEMS FOR UVC LIGHT SOURCE TEMPERATURE AND FUNCTION IN SANITIZING DEVICE

(76) Inventor: Ryan J. Douglas, Stillwater, MN (US)

(21) Appl. No.: 13/078,504

(22) Filed: Apr. 1, 2011

(51) Int. Cl. A47L 9/28; A61L 2/10; B23P 11/00

(52) U.S. Cl. 15/339; 29/428; 96/224

(57) ABSTRACT

Presented herein are various techniques for cooling a UV light source and keeping it within a predetermined temperature range. One embodiment is directed to using Venturi pump for the thermal control. Another embodiment employs a mechanical or electrical self-adjusting valve. Another embodiment is directed to using a forced air system to actively blow air across the light source. Examples of the device are an air cleaner, a vacuum cleaner, a mobile floor-sanitizer, and a hand-held sanitizer.

### TECHNICAL FIELD

The Technical Field is directed to systems for operating ultraviolet (UV) bulbs, for instance, UV bulbs in vacuum cleaners and appliances.

### BACKGROUND

Ultraviolet (UV) light has been proposed for use in vacuum cleaners for many years, for example in U.S. Pat. Nos. 2,648,396, 2,632,912, 3,975,790, 4,907,316, and 5,233,723. More recent interest in this area is reflected by, for example, U.S. Pat. No. 6,968,595, US2006/0185116, US2006/0185117, US2006/0278088, US2007/0209144, US2007/0209147, and US2007/0067943. Despite this interest, this technology has largely not been adopted by consumers.

### SUMMARY OF THE INVENTION

Presented herein are techniques for operating a UV light source without excessivecooling, while keeping the light source within a predetermined temperature range. One embodiment is directed to using a Venturi pump for the thermal control. Another embodiment employs a mechanical or electrical self-adjusting valve. Another embodiment is directed to using a forced air system to actively blow air across the light source. Examples of the device are an air cleaner, a vacuum cleaner, a mobile floor-sanitizer, and a hand-held sanitizes.

One embodiment is directed to a device for sanitization or sterilization with an ultraviolet light source comprising an ultraviolet light source in an area of the device that is at least partially enclosed that comprises a self-adjusting valve for adjusting air flow through the area, wherein the valve is adjustable to change a flow of air through the valve in response to a change in ambient temperature, bulb temperature, pressure, or air flow.

Another embodiment is a method for sanitization or sterilization with an ultraviolet light source comprising flowing air across an ultraviolet light source in an area that is at least partially enclosed that comprises a self-adjusting valve for adjusting air flow through the area, wherein the valve is adjustable to change a flow of air through the valve in response to a change in temperature, pressure, air flow, or electric current provided to the light source.

Another embodiment is a vacuum cleaner that creates a suction that draws dust and other particles from a surface to be cleaned through a suction path into a container, the cleaner comprising a Venturi pump that pulls cooling air across an ultraviolet light source to maintain the light source within a predetermined minimum and maximum temperatures, e.g., with a minimum of about 45° C. and a maximum of about 100° C., wherein the light source provides ultraviolet light on the surface that is to be cleaned.

Another embodiment is a method of cooling an ultraviolet light source in a vacuum cleaner having a motor that creates a suction that draws dust and other particles from a surface to be cleaned through a suction path into a container comprising pulling air across the light source with a Venturi pump comprising a narrowed portion in the suction path and an opening at or near the narrowed portion, with the opening providing for air to flow across the ultraviolet light source and through the opening and into the suction path, wherein the ultraviolet light source is positioned to shine ultraviolet light on the surface to be sanitized.

Another embodiment is a vacuum cleaner that creates a suction that draws dust and other particles from a surface to be cleaned through a suction path into a container, the cleaner comprising an adjustable valve that adjusts control of a flow of air across an ultraviolet light source in response to a change in temperature, pressure, air flow, or electrical current.

Another embodiment is an air cleaner that draws air into the cleaner, directs ultraviolet light from an ultraviolet light source into air that is drawn into the cleaner, and passes the air out of the cleaner, the cleaner comprising a self-adjusting valve that automatically adjusts the flow of air into the cleaner or out of the cleaner.

Further embodiments relate to a thermal control system that controls a temperature using a thermoelectric cooler and/or a heat sink. All of these thermal control systems and features may be used together or in combination and may be directed to various devices. Examples of the devices are air cleaners (stationary or mobile), mobile sanitizers, vacuum cleaners equipped with an ultraviolet light source, and hand-held sanitizers.

### BRIEF DESCRIPTION OF THE FIGURES

FIG. 1 is a schematic cross-sectional view of a Venturi pump cooling a light source;

FIG. 2A is a perspective view of a vacuum cleaner head;

FIG. 2B is a bottom plan view of the head of FIG. 1A;

FIG. 2C is a partial cross sectional view along the line C-C′ of FIG. 2B;

FIG. 2D is a partial cross sectional view along the line D-D′ of FIG. 2B;

FIG. 2E is a cross sectional view of a portion of the head along the line E-E′ of FIG. 3A;

FIG. 3A depicts an embodiment of a self-adjusting air flow valve;

FIG. 3B depicts an alternative embodiment of a self-adjusting air flow valve;

FIG. 4A is a front elevated view of an embodiment of a self-adjusting air flow valve that employs a plurality of leaflets;

FIG. 4B is a side perspective view of the valve of FIG. 4A;

FIG. 5A is a plan view of a self-adjusting valve in a first position;

FIG. 5B is a plan view of the valve of FIG. 5A is a second position that is relatively more closed;

FIG. 6 is an elevated view of a self adjusting valve;

FIG. 7 depicts an alternative embodiment of FIG. 2 that employs one or more electrically controlled valves;

FIG. 8 depicts an alternative embodiment of FIG. 2 that employs an electrically controlled forced air system;

FIG. 9 depicts an air treatment system that substantially encloses a light source and has a self-adjusting valve and forced air system; and

FIG. 10 is an alternative embodiment of the embodiment of FIG. 2, and depicts a heat sink or heat pump thermal control system.

### DETAILED DESCRIPTION OF THE PREFERRED EMBODIMENTS

Presented herein are thermal control systems for cooling a UV light source without excessive cooling, and keeping the light source operating within a predetermined temperature range. The control system may employ a Venturi pump to move air across a light source. Other control system embodiments provide a self-adjusting valve that dynamically regulates air flow to maintain the temperature. Some valves are mechanically self-adjusting, while other valves are electronically self-adjusting by use of electronic sensors and/or controls to detect an environment of the base and/or bulb and make adjustments as needed for thermal control. Other thermal control systems employ a heat sink or a thermoelectric cooler (TEC). The light sources and control systems can be part of a variety of devices, including vacuum cleaners, sanitizers, and air treatment machines.

One embodiment provides for a thermal control system providing a cooling effect over a UV or other light source by use of a Venturi pump. A Venturi pump involves a fluid flow through a conduit that has a narrowing point. At the narrowing point is an opening in the conduit. The term “at” in this context means that the opening is close enough to the narrowing point to benefit from the Venturi effect. The fluid accelerates as it passes through the narrowing point and, because of the Venturi effect, its pressure decreases. This pressure drop is used to draw another fluid into the channel. The pump may be employed by passing air through the conduit and placing an opening at a narrowing point. The opening is used to pull air through an airway (path) that includes at least a portion of a light source.

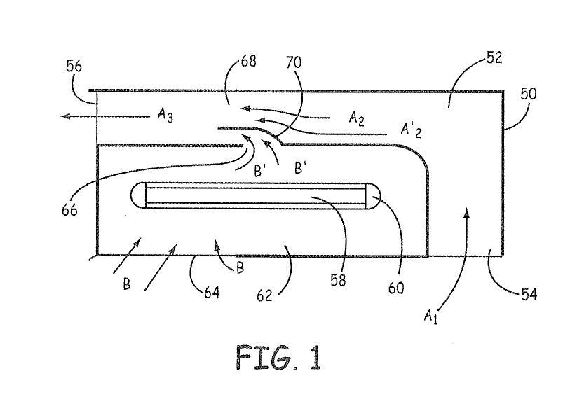

FIG. 1 is a cross-sectional view showing an example of a Venturi pump for cooling a light source. Housing 50 provides a conduit 52 having an entrance 54 and exhaust 56, with an airflow passing through conduit 52 driven by a fan (not shown) or other means. Light source 58 mounted in light housing 60 is in partially enclosed area 62 that has entrance 64 and exhaust 66. Exhaust 66 is disposed at narrowing portion 68 of conduit 52, behind wall 70. Flowing air passes into conduit 52 as indicated by arrow A₁, accelerates at narrowing point 68 as indicated by arrows A₂, A₂′, and exits via exhaust 56. Air is pulled into area 62 through opening 64 as indicated by arrows B and is exhausted through exhaust 66 as indicated by arrows B′, with the acceleration of the air in the main conduit driving flow across light source 58 by the Venturi effect. The light source is in a partially enclosed area (a partial enclosure) but is not fully enclosed. A light source that is substantially enclosed is effectively in a container that contains effectively all of the light emitted by the source; for instance, an air cleaner or vacuum cleaner with an internal UV light. A light source that is about half enclosed has walls or the like around about half of its circumference: for instance, a light set in a hemispherical light recess. Lights are often at least partially enclosed by placement in a lighting recess.

Embodiments of a thermal control system directed to flowing air across a light source can be practiced with the air flowing across a bulb that is the light source and/or a base that provides power to the source. Air may be passed specifically over the base and not the bulb, or vice versa. The design choice for drawing heat from one or both locations provides options for regulating temperatures.

An embodiment of a Venturi pump thermal control system is a vacuum cleaner that has a motor that intakes air and dirt or other materials through a main conduit. A light source in the vacuum cleaner is not in the main conduit. Instead, the light source is in an at least partially enclosed area that is exhausted by an air path that is part of the Venturi pump. The air path can be a tubular conduit or have other shapes, and is determined by a pressure gradient driven by the pump that pulls air through the path. Alternatively, a light source may be placed in the canister, bag, dirt collection container, or other portion of the vacuum cleaner to illuminate areas besides the surface to be cleaned.

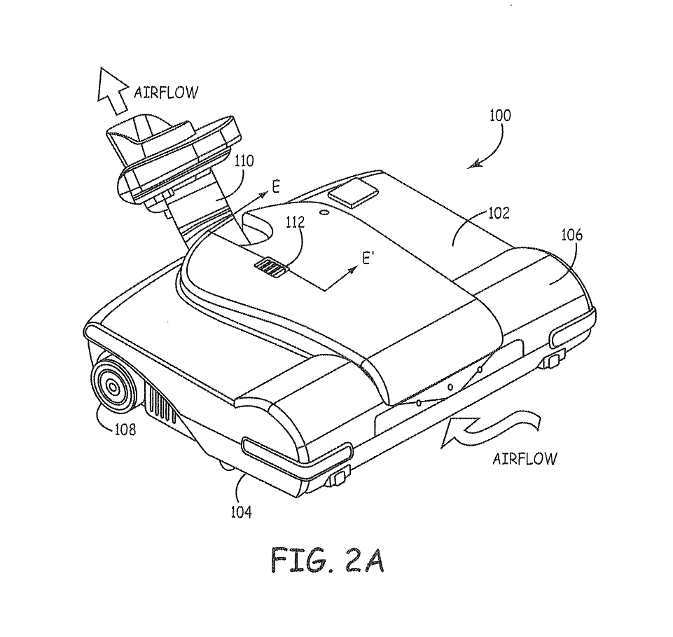

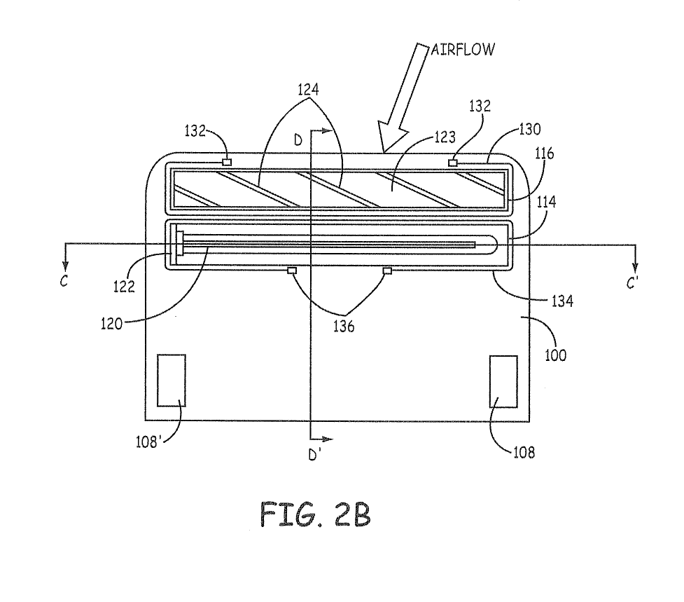

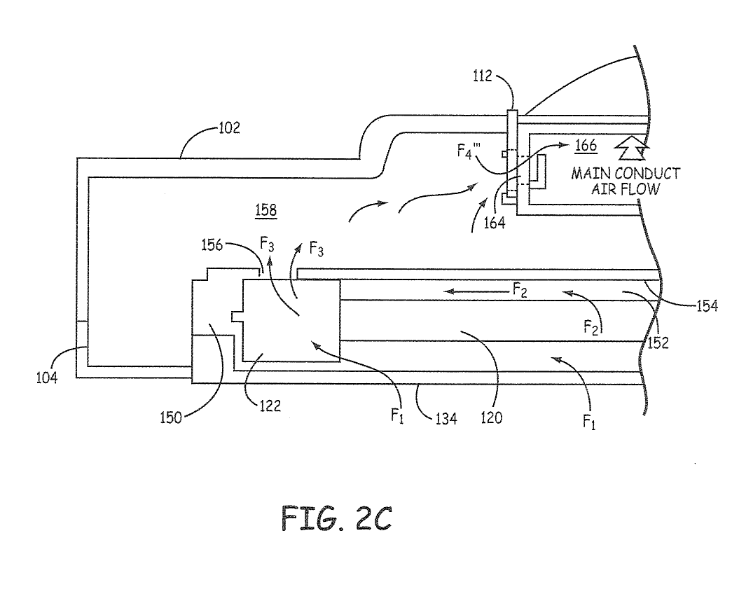

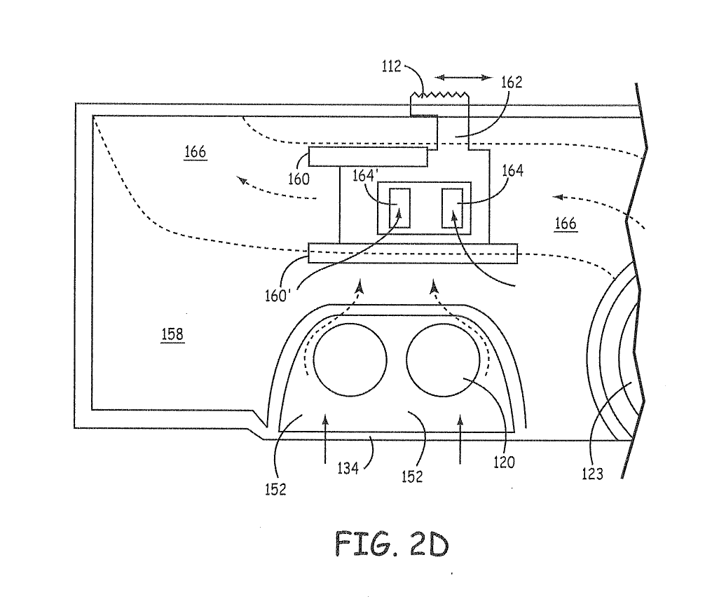

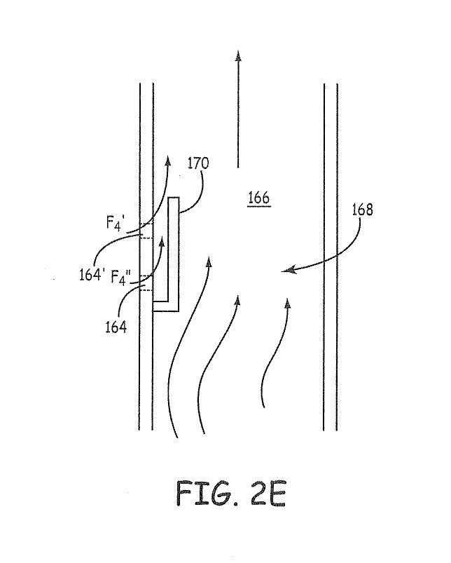

FIGS. 2A to 2E illustrate such an embodiment. FIG. 2A depicts vacuum cleaner head 100 having outer housing upper member 102 and outer housing lower member 104, with the members 102, 104 cooperating to provide enlarged roller casement area 106. Wheel 108 and counterpart wheel 108′ (not shown) are mounted on head 100. Adjustable siphon 110 receives a conduit from a vacuum source (not shown), for instance a canister vacuum cleaner. Manual air flow control 112 extends through outer housing upper member 102. A bottom view of head 100 is provided in FIG. 2B, with lower housing member 104 providing light source opening 114 and roller brush opening 116. Ultraviolet C (UVC) light source 120 on light source base 122 is mounted inside the outer housing. Roller 123 with brushes 124 is mounted so that brushes 124 may contact a cleaning surface. Roller access cover 130 is attached by hinges 132 to member 104. Light source access cover 134 is attached by hinges 136 to member 104. As is familiar with domestic vacuum cleaners, the vacuum source creates a low pressure across roller 123 and pulls dust and other materials through the vacuum head and into a collection container, as indicated by the arrows labeled Airflow in FIGS. 2A and 2B. FIG. 2C is a cross-section of FIG. 2A that depicts this same airflow through the conduits with the arrow labeled main conduit airflow.

FIGS. 2C and 2D also depict the flow of air across base 122 of UVC source 120. Base 122 is mounted in mount 150 that is secured (not shown) inside the outer housing. UVC source 120 is disposed in reflection chamber 152 having surfaces 154 made or lined with reflective material. The choice of a reflective material is known to artisans, for instance, polished aluminum, stainless steel, mirrors, and reflectors. Chamber 152 has light source opening 114 and opening 156 near base 122. Outer housing members 102, 104 contain housing inner space 158. Brackets 160, 160′ support movable cover 162 that is integral with air flow control 112. Air passages 164, 164′ connect inner space 158 with main conduit 166.

A Venturi pump in the main conduit pulls air across the light source and its base, through the inside of the housing, and into the main conduit, as depicted at FIGS. 2D to 2E. Suction pulls air through main conduit 166 and past narrowing portion 168 across from wall 170. Arrows F₁ depict air flowing into reflection chamber 152, then arrows F₂ show flow across source 120 and base 122, and arrows F3 show flow out of chamber 152 through opening 156 into interior space 158. Flow from space 158 proceeds along arrows F4′, F4″, F4′″ through openings 164, 164′ and into main conduit 166.

A user may manually move flow control 112 to shift cover 162 to partially or completely cover openings 164, 164′. Other embodiments provide for automated movement of the same by means of a sensor that detects a change, as per other description herein. Flow is thus adjustable by opening-up or closing the air flow path across the base and/or light source. The vacuum may be operated at various speeds by a user to provide different amounts of suction; as the suction changes, flow through the light chamber is affected.

It is generally recognized that cooling a UV source is important to prevent it from failing by over heating. It is conventionally not appreciated, however, that too much cooling can cause a UV source to underperform, or that this underperformance is a significant obstacle in device performance. Further, it is conventionally not appreciated that overheating can not only cause bulb failure, it can also cause the bulb's output spectrum to drift, particularly with respect to UVC sources. This drift tends to compromise sanitizing performance, so that it is desirable to improve how, and how well, light source temperatures are controlled. Controlling the temperature only within the range required to optimize bulb life or prevent failure is not an adequate temperature for achieving suitable UVC sanitization performance. Experiments with UVC light sources in sterilizing applications indicate that about 45° C. to about 100° C. is a useful range for control; artisans will immediately appreciate that all the ranges and values within the explicitly stated ranges are contemplated, e.g., about 45° C. or about 45° C. to about 70° C. The light source may be allowed to warm up to reach the lower end of the range, about 45° C. and then cooled to limit the upper temperature, but without cooling below the predetermined value, e.g., 45° C.

An embodiment of the invention is a vacuum cleaner equipped with a UVC source that provides at least 20 milliWatts per square centimeter (mW/cm²) UVC light at a surface to be cleaned throughout a time of light source operation after the device is warmed-up and thereafter while the device is operated. The UVC source may be operated with an output of, for example, more than about 20 mW/cm², e.g., 35 mW/cm² or 40 mW/cm², or between about 20 mW/cm² and 60 mW/cm²; artisans will immediately appreciate that all the ranges and values within the explicitly stated ranges are contemplated. Further, the UVC bulb may be maintained at a temperature of between about 45° C. to about 100° C. The UVC output determines how long it takes to achieve a microorganism kill rate. A suitable dosage is 40 mJ/cm² at the surface that is being sanitized.

Embodiments of the device include a vacuum or other mobile device equipped with a UV source with a UV output intensity having a temperature and an intensity of UV source output that is within the stated ranges over a time period starting 5 minutes after turning on the light source until 30 or 60 minutes of continuous operation of the light source have elapsed. An embodiment is, for instance, such a devices with a UV source having an intensity of at least 20 mW/cm2 or at least 35 mW/cm2 that may be maintained between a temperature of about 45° C. and about 80° C. (or alternatively about 100° C.) throughout a time period starting at 5 minutes and ending at 60 minutes of continuous operation of the UV source.

In the case of a UV light source combined with a vacuum cleaner that involves the vacuum's suction in the cooling process, significant design effort is required to maintain the target temperature range throughout a user's cleaning process. Maintaining light source temperatures within the target range is made more difficult if a variable speed suction is provided (e.g., with a variable speed motor) or a plurality of vacuum suction set points are provided to the user, since more suction drives more air flow which creates more cooling, which can force the bulb out of the bottom of the target range.

A vacuum, however, may be equipped with a Venturi pump system or other system as described herein that provides better control of temperature. An advantage of these various approaches is that a user's choice of a motor speed to drive the suction does not so proportionally affect flow of air across a bulb or bulb cooling system so as to move the bulb outside of a predetermined temperature range. Controlling the temperature provides improved performance. Further, these other systems are readily adapted to use of an adjustable air flow choke that limits the air flow across the light source. A plurality of user-controlled suction settings can thus be more readily designed into the device. Accordingly, embodiments include vacuum cleaners as set forth herein with a range of air flow that provides suction that varies by a factor of between about 1.5 to about 5; artisans will immediately appreciate that all the ranges and values within the explicitly stated ranges are contemplated. A factor of 2 refers to a user control that allows air flow for suction to be doubled, e.g., as measured in cubic feet per minute.

A light source may be an ultraviolet light (UV) source, e.g., ultraviolet A (UVA; about 400 nm to about 315 nm), ultraviolet B (UVB; about 315 nm to about 290 nm), ultraviolet C (UVC; about 290 nm to about 100 nm). UVC can be found in artificial sources such as mercury arc lamps and germicidal lamps. Light sources commonly referred to as UVC lamps can be used, e.g., as in the VERILUX TRAVEL WAND, which is a commercially available sterilization wand. Some light sources are referred to as high pressure UVC lamps, and typically have a peak at 254 nm. UVC sources may further provide a secondary peak at about 185 mm Medium pressure UVC lamps vary somewhat and typically have multiple peaks from abort 225 nm to about 600 nm Another light source embodiment is a mixture of UVA, and/or UVB, and/or UVC light in the range of about 185 nm to about 365 nm. The light may come from a filtered broad spectrum light source to provide a spectrum of light within the 185-365 range, or a plurality of light sources may be used that each provide at least one peak within the 185-365 range. For instance, two or three LED light sources may be used. Moreover, the light source may exclude wavelengths outside of the 185-365 range.

The light source may be mounted according to the style of vacuum cleaner to illuminate the surface being vacuumed, e.g., canister or upright, see for example U.S. Pat. No. 2,632,912, U.S. Pat. No. 4,907,316, US 2006-0185116, US 2007-0192986 each of which are hereby incorporated by reference herein to the extent they do not contradict what is explicitly disclosed herein. Alternatively, the light may be mounted in other positions to sanitize material drawn into the device or materials that have been captured in the device, for instance: in the canister, in the collection container, in the suction tube, in the main conduit, in a position to illuminate one or more of the same. Other features and options described herein may further be incorporated into these vacuum devices.

A Venturi pump for controlling a temperature across a light source may similarly be applied to a sanitizing device, for example as in U.S. Pub 2010/0104471 which is hereby incorporated by reference for all purposes to the extent it does not contradict what is explicitly disclosed herein. And, for instance, a fan or motor to move air may be incorporated into the device to move air through a conduit that has an opening that fluidly communicates with an air path across all or a portion of the light source. Alternatively, the fan or motor may be used to push or pull air directly across the device. Moreover, an air purifier that uses a UV light source to treat air may incorporate one or more of these features.

A self-adjusting valve may be used to control an air flow across a UV light source in a device. In one embodiment, the self-adjusting valve is substituted for the opening at the Venturi and/or the narrowing portion of the Venturi pump is eliminated in the conduit. Thus the self-adjusting valve may be placed at exhaust 66 of the embodiment of FIG. 1; narrowing portion 68 may remain in place or be removed.

A self-adjusting valve may be mechanically or electronically adjustable. A mechanically self-adjusting valve is affected by a change in its environment so that it moves and changes flow through the valve. Examples are biased members or thermally sensitive members that move in response to a change in pressure or temperature. An electronically self-adjusting valve refers to a valve that uses electronic data to control a valve position. Examples are electronic sensors or transponders in combination with a microcontroller that directs a controller to adjusts a valve position. A movement of the valve may be set to control an air flow to regulate temperature of a light source.

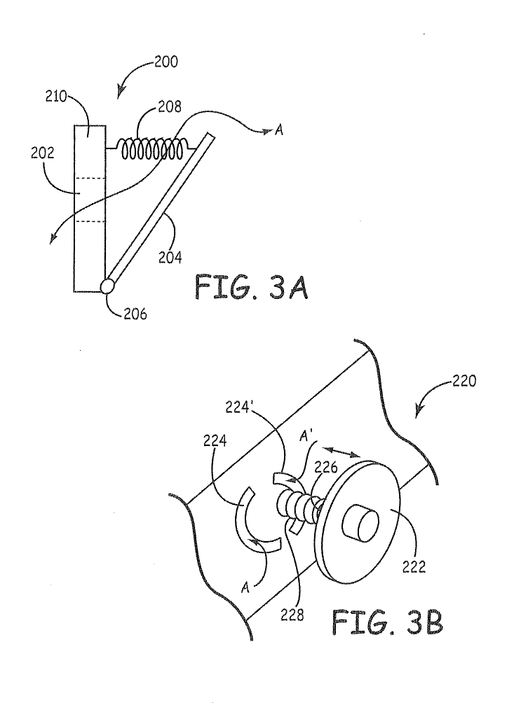

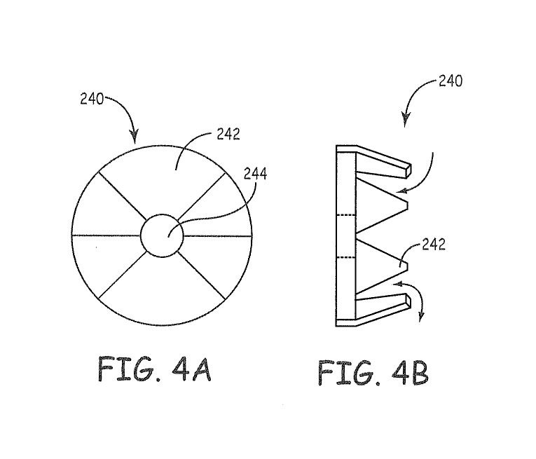

FIG. 3A depicts a thermal control system that comprises a mechanical self-adjusting valve. Valve 200 has passageway 202 coverable by cover 204 mounted by hinge 206 and biased towards the opening position by spring 208 that is attached to valve body 210 and cover 204. Air, indicated by arrow A, flows beneath cover 204 and through passageway 202. As the air flow increases, cover 204 is forced against spring 208 and tends to move towards a closed position, thereby restricting airflow. FIG. 3B depicts an alternative embodiment of a mechanical self-adjusting valve. Valve 220 has openings 224, 224′ coverable by cover 222 mounted by post 226 and biased towards an open position by spring 228 that is wrapped around post 226. Air, indicated by arrows A, A′ flows through openings 224, 224′. As the air flow increases, cover 222 is forced towards openings 224, 224′ to reduce flow. FIGS. 4A and 4B depict mechanical self-adjusting valve 240 having a plurality of leaflets 242 that move to adjust the size of air passage 244. Leaflets 242 are molded from a material with a stiffness that biases them to remain in an open position. As air flow through passage 244 increases, the leaflets are forced downwardly and reduce the flow of air. Many mechanically self-adjusting valves are known. Valves include, for example, ball valves, butterfly valves, disc valves, check valves, choke valves, diaphragm valves, gate valves, globe valves, knife valves, needle valves, pinch valves, position valves, plug valves, poppet valves, spool valves, and expansion valves.

In use, a mechanical self-adjusting valve that responds to pressure may be placed over an opening that regulates an air flow across a light source. An increase in flow quantity and/or speed across the valve changes the pressure. In a first case an airflow may be drawn into an area at least partially enclosing a light source, across the source, and then drawn out of an exhaust. The drawing force may be a fan or motor, for example. The valve will tend to close as the drawing force increases; in this manner, the valve is more open when flow is low and relatively more closed as flow is progressively increased. In a second case, a fan or other means pushes an airflow through an inlet opening governed by an adjustable valve and into an area that at least partially encloses a light source. The valves may thus be placed at an inlet and/or an outlet.

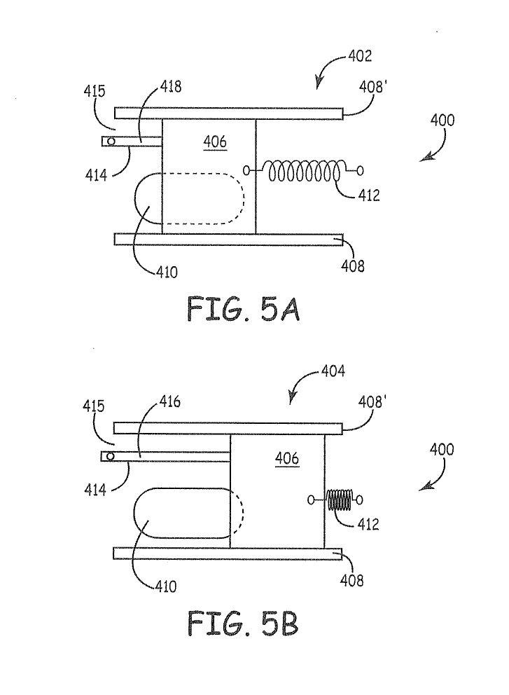

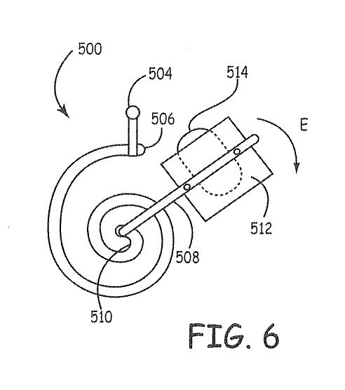

A further example of a thermal control system made with a mechanically self-adjusting valve is a valve that is moved by a thermal actuator. The actuator has a coefficient of thermal expansion that provides expansion or contraction in response to a change in temperature that is large enough to change the actuator's dimensions so that it can move the valve's position. The thermally responsive material may be, for example, a metal, metal alloy, or hard plastic material. Examples of metals and metal alloys include one or more of aluminum, brass, bronze, chromium, copper, gold, iron, magnesium, nickel, palladium, platinum, silver, stainless steel, tin, titanium, tungsten, zinc, zirconium, and the like. Pairings of materials to make a bimetallic strip are known. FIGS. 5A and 5B depict valve 400 in a first closed position 402 and a second open position 404. A valve that is completely shut is referred to as fully closed. Cover 406 moves within tracks 408, 408′ over passageway 410. Spring 412 tends to move cover 406 to a closed position. Thermal actuator 414 mounted on face 415 responds to a change in temperature by expanding (416) or contracting (418). Expansion of actuator 414 moves the valve towards open position 404. FIG. 6 depicts self-adjusting thermally actuated valve 500 that comprises coiled bimetallic strip 502 that is fixed by fastener 504 at outer coiled end 506 and has post 508 mounted in central coiled area 510. Cover 512 is mounted to post 508 and moves relative to opening 514. Arrow E depicts movement of cover 512 in response to an increase in temperature. Changes in length of strip 502 move post 508 to move cover 512 to provide for a greater or lesser covering of opening 514.

Thermally self-adjusting valves may be placed over an opening that regulates an air flow across a light source or portion thereof. The opening may be an inlet and/or an exhaust of an area that is at least partially enclosed. The air may be forced into, or pulled out of, the area. An actuator portion of the valve may be placed at or near the light source so that temperature changes at the source are communicated to the actuator. In one embodiment the thermally responsive actuator is within 0.1 to 5 cm of the bulb and/or the light source base; artisans will immediately appreciate that all the ranges and values within the explicitly stated ranges are contemplated. In another embodiment, the actuator is within the same enclosure as the bulb and/or base.

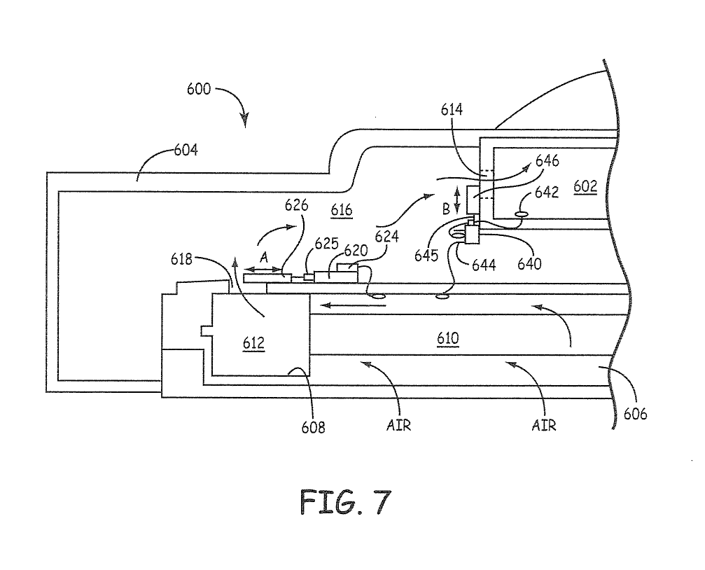

A thermal control system that comprises an electronically self-adjusting valve may be used to regulate a temperature of a light source. FIG. 7 depicts vacuum cleaner head 600 having a main suction conduit 602, housing 604, and a partially enclosed area 606 that contains light source 608 having bulb 610 and base 612. Air flowing through main conduit 602 pulls air in through opening 614 from housing interior 616, which in turn draws air through opening 618 that serves as the exhaust for area 606. Air flows into area 606 from the surroundings, as indicated by arrows labeled Air. Electronically self-adjusting valve 620 has sensor 622 disposed in area 606 electrically connected to controller 624 that controls motor 625 to actuate gate 626 to open or close opening 618, as indicated by arrow A. Additionally, but alternatively and optionally, electronic self-adjusting valve 640 has sensor 642 disposed in conduit 602 and/or sensor 643 disposed in area 606 electrically connected to controller 644 that controls motor 645 to actuate gate 646 to open or close opening 614, as indicated by arrow B. The controllers are programmed to sense a temperature, pressure, or air flow velocity and adjust the area available for a flow of air through the opening that they govern. FIG. 7 uses a vacuum cleaner as an example.

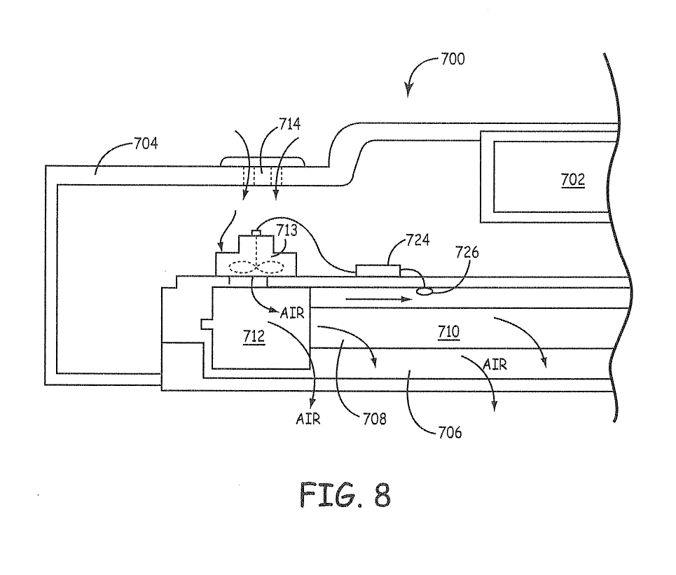

FIG. 8 depicts a forced-air thermal control system for regulating a temperature of a light source. A set of electronic controls are integrated with the system to maintain the source within a predetermined temperature range. FIG. 8 depicts vacuum cleaner head 700 having a main suction conduit 702, housing 704, and a partially enclosed area 706 that contains light source 708 having bulb 710 and base 712. Air driven by fan 713 pulls air in through opening 714 that is forced into area 706 and out of the device, as indicated by arrows labeled Air. Fan 713 is controlled by controller 724 electrically connected to sensor 726 disposed in area 706. Sensor 726 provides temperature data to controller 724 that controls speed of fan 713.

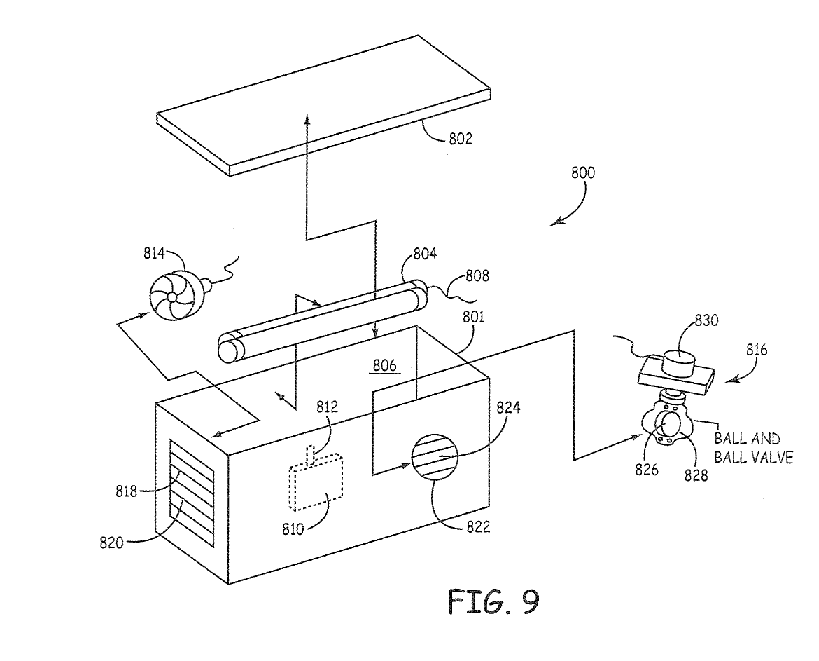

Certain of the examples are directed to vacuum cleaners. Other systems may be similarly adapted to use the thermal control systems, including UV sanitization devices and air treatment devices. FIG. 9 is an example of an air treatment device, with the self-adjusting valve being depicted as an electronically controlled ball valve. Air treatment device 800 has bottom housing portion 801 that joins with lid 802. UV light source 804 is mounted on an interior wall 806 of housing 801. The source is electrically connectable to a power source via power cord 808 that extends through the housing (only partially shown). Controller 810 is mounted inside housing 801 and is electrically connected (now shown) to sensor 812, fan 814, and valve 816. Fan 814 is mounted inside housing 801 and over opening 818 that is overlain by a plurality of baffles 820. Valve 816 is mounted inside housing 801 over opening 822 that is covered by a plurality of baffles 824. Valve 816 has ball 826 in ball housing 828 and actuator 830 that moves the ball to open and close valve 816. Air is forced into housing 801 and exposed to UV source 804. Valve 816 controls air flow out of the housing. Controller 810 controls fan 814 and/or valve 816 according to temperature or other data provided by sensor 812 to control light source 804 within a predetermined temperature range, e.g., 45° C. to 100° C. The controller may further control power to the light source. The fan may be run in either direction so that air is pulled through, or pushed into, housing 801 through egress 818. Other valves may be provided as alternatives to a ball valve. The fan and valves may be mounted inside or outside the housing and other features may be added to the housing, e.g., filters, air fresheners, and ionization sources. Other air treatment devices may be adapted for use with embodiments disclosed herein, as in US Pub. No. 2006/0057020, U.S. Pat. Nos. 5,925,320, 6,264,888, 6,497,840, 6,494,940, 6,949,223, each of which are hereby incorporated by reference for all purposes to the extent they do not contradict what is explicitly disclosed herein.

The thermal control systems may comprise sensors. The sensors sense an environmental condition, or a change therein. Examples of sensors are air flow sensors, pressure sensors, temperature sensors, and sensors that detects a voltage or a flow of current. For instance, a flow of current to the light source is correlated to the light and heat it produces. A sensor that monitors the current can be used to adjust a controller, valve, or cooler. The coordination of sensors with control of a device are known, and artisans reading this specification will be able to use a sensor in a control system to control an environment of a light source and its temperature range.

A thermal control system may also comprise a thermoelectric cooler (TEC), also known as a Peltier cooler. A TEC is a solid-state heat pump: when direct current runs through it, heat is moved from one side to the other. Therefore the TEC can be used as a temperature controller that either heats or cools. TECs are typically constructed using two dissimilar semi-conductors, one n-type and the other p-type (they are different because they need to have different electron densities in order for the effect to work). The two semiconductors are positioned thermally in parallel and joined at one end by a conducting cooling plate (typically copper or aluminum). A voltage applied to the free ends of two different conducting materials results in a flow of electricity through the two semiconductors in series. The flow of DC current across the junction of the two semi-conductors creates a temperature difference. As a result of the temperature difference, Peltier cooling causes heat to be absorbed from the vicinity of the cooling plate, and to move to the other (heat sink) end of the device.

The TEC may be coupled with a sensor and controlled by a microprocessor, with data from the sensor providing a basis for operation of the TEC to cool or heat a light source. Alternatively, the TEC may be controlled by a timer that tracks cumulative light source operating time; for example, by providing heat during a warm-up time period and providing cooling after a set time. The TEC may also be driven in a manner proportional to current that drives the light source, either alone or in combination with other sensors, microprocessors, and controls. For instance, a TEC may be turned on after a warm-up period has passed and then run in a cooling mode using a current that is the same as, or proportional to, the current passing through the light source. In such embodiments, the operation of the light source and the TEC may be matched to provide a steady-state temperature effect, with the amount of heat generated by the light being substantially drawn off by the TEC.

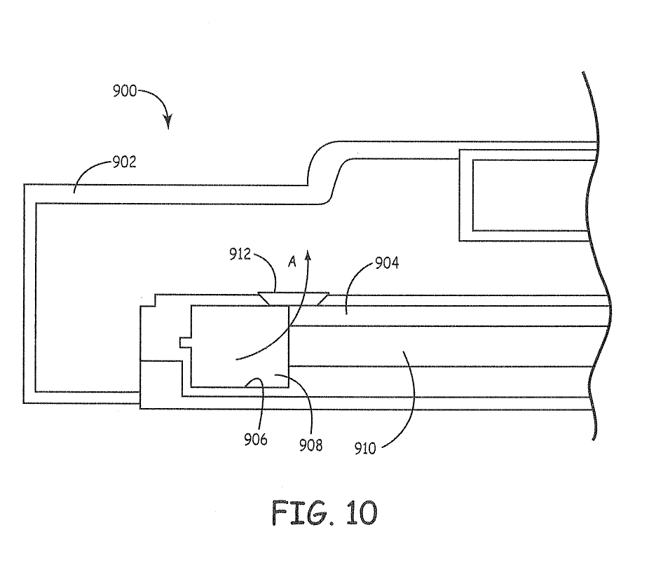

FIG. 10 depicts a thermal control system comprising a thermoelectric cooler (TEC). Vacuum cleaner head 900 has outer housing 902 that accommodates light recess 904 that houses light source 906 having base 908 and bulb 910. TEC 912 is disposed near base 906. The TEC may be operated in a cooling mode to draw heat from source 906 as indicated by arrow A. The TEC is electrically connected to a power source (not shown), as is light source 906.

A thermal control system may also comprise a heat sink for thermal transfer. An example of a heat sink is copper or aluminum, or other effective heat-conductor. In this embodiment, a block of metal or other conductor is disposed at or near a base of the light source to draw heat away from the same. A portion of the heat sink is disposed in an area that allows for heat dissipation, e.g., air outside of the device, or an interior space. For instance, the TEC of FIG. 10 may be replaced by a heat sink. A heat sink may be used by itself or in combination with other thermal control systems.

Mobile sanitizing devices or hand-held sanitizers may be adapted for use with thermal control systems as set forth herein. For instance, US Pub. Nos. 2010/0104471 and 2008/0260601 describe a rolling UVC sanitizing system and hand-held wand systems. Such a system may include a fan or other means for forcing air across the light source or to use air to create a Venturi pump effect, as well as self-adjusting valve(s) in combination with the same. Similarly, a wand disclosed therein may be adapted to be cooled in the same fashion. These devices may use these features and other features described herein for other systems, e.g., air cleaners and/or vacuum cleaners.

Indicators may be used with a device described herein to indicate a light source temperature or temperature status to a user, with the device comprising a user-adjustable control or valve to make adjustments to control the temperature. For instance, a panel display and/or an audio signal may indicate a temperature status of the light source. The term temperature status means the temperature of the source (base and/or bulb), or a condition of the source temperature being above and/or below and/or within the temperature range. For instance, a light could indicate yellow, red, and green as a status of being below, above, or within, respectively, a desired temperature range. A user may then make an adjustment, e.g., increasing an air flow, opening a valve, or increasing/decreasing a fan speed. A valve or system as set forth herein may also be used or adapted to be a user-controllable valve in combination with such indicators. The term indicator is broad and includes a display and an audio signal. The term display is broad and includes, e.g., lights, light arrays, liquid crystal displays, and video displays. A display may be augmented with an audio signal, depending on the overall functionality of the display.

Patents, patent applications, and publications set forth herein are hereby incorporated by reference herein to the extent they do not contradict what is explicitly disclosed herein. The embodiments describe a variety of features. In general, the features may be mixed-and-matched to make other embodiments as guided by the need to make a functional device.

1. A vacuum cleaner that creates a suction that draws dust and other particles from a surface to be cleaned through a suction path into a container, the cleaner comprising an ultraviolet light source and thermal control system that maintains the light source at a minimum temperature of about 45° C. and a maximum temperature of no more than about 100° C.

2. The cleaner of claim 1 wherein the light source provides ultraviolet light on the surface that is to be cleaned.

3. The cleaner of claim 1 wherein the ultraviolet light source comprises an ultraviolet C light source.

4. The cleaner of claim 1 wherein the vacuum comprises a variable speed motor to produce a variable suction.

5. The cleaner of claim 1 with the thermal control system comprising a Venturi pump that pulls cooling air across the ultraviolet light source, wherein the Venturi pump comprises a narrowing portion that is disposed within the suction path and the cooling air enters the suction path at the narrowing portion.

6. The cleaner of claim 5 wherein the cooling air path comprises a flow path across a base of the light source, into an interior of a head of the vacuum cleaner, and through an opening that connects the interior to the suction path.

7. The cleaner of claim 6 wherein the suction flow path comprises a flow path across a roller brush disposed in the head of the vacuum cleaner and a conduit that connects the head to a motor that provides the suction.

8. The cleaner of claim 7 wherein the opening that connects the interior of the head to the suction path has a cross-sectional area that is adjustable by a user.

9. The cleaner of claim 1 with the thermal control system comprising a self-adjusting valve for adjusting an air flow across the light source.

10. The cleaner of claim 9 wherein the valve is adjustable to change a flow of air through the valve in response to a change chosen from the group consisting of temperature, pressure, air flow, and electrical current drawn by the light source.

11. The cleaner of claim 9 wherein the valve is adjustable to change a flow of air through the valve after a predetermined total time of operation.

12. The cleaner of claim 9 wherein the valve is a mechanically self-adjusting valve.

13. The cleaner of claim 9 comprising an electrical control to adjust the valve.

14. The cleaner of claim 1 wherein the thermal control system comprises a thermoelectric cooler.

15. The cleaner of claim 1 further comprising an indicator that indicates a temperature status of the light source.

16. A device for sanitization or sterilization with an ultraviolet light source comprising an ultraviolet light source and a self-adjusting valve adjustable to change a flow of air through the valve in response to a change chosen from the group consisting of temperature, pressure, air flow, and electrical current drawn by the light source.

17. The device of claim 16 being chosen from the group consisting of an air cleaner, a mobile floor-sanitizer, and a hand-held sanitizer.

18. The device of claim 16 wherein the self-adjusting valve increases and/or decreases the air flow to maintain a predetermined temperature range of the light source at a minimum temperature of at least about 45° C. and a maximum of no more than about 100° C.

19. The device of claim 18 wherein the valve is a mechanically self-adjusting valve.

20. The device of claim 19 comprising an electrical control to adjust the valve.

21. The device of claim 20 wherein the valve is actuated by an electrically powered actuator controlled by a controller that receives data from a sensor chosen from the group consisting of an air flow sensor, a pressure sensor, a temperature sensor, and a sensor that detects a flow of current to the light source.

22. The device of claim 16 further comprising a motor that pushes or pulls air across at least a portion of the light source and through the valve.

23. The device of claim 16 further comprising an indicator that indicates a temperature status of the light source.

24. The device of claim 23 wherein the valve is adjustable by a user in response to the temperature status.

25. The device of claim 16, wherein the device is an air cleaner that draws air into a compartment that comprises the light source, directs ultraviolet light from the source onto the air.

26. A process of making a sanitization device comprising providing an ultraviolet light source and a thermal control system to maintain the light source at a minimum temperature of about 45° C. and a maximum temperature of no more than about 100° C.

27. The process of claim 26 further comprising providing an indicator to indicate a temperature status of the light source.

28. The process of claim 27 wherein the thermal control system comprises a member chosen from the group consisting of a heat sink, an adjustable valve, a Venturi pump, and a thermoelectric cooler.

29. The process of claim 28 wherein the device comprises a member chosen from the group consisting of a vacuum cleaner, a mobile sanitizer, a hand-held sanitizer, and an air cleaner.
